(19) United States
(12) Patent Application Publication
(10) Pub. No.: US 2009/0182305 A1
(43) Pub. Date: Jul. 16, 2009

(54) Reusable Cannula

(76) Inventor: Kelly Tjelmeland, Austin, TX (US)

Correspondence Address: DUBOIS, BRYANT, CAMPBELL & SCHWARTZ, LLP, 700 LAVACA STREET, SUITE 1300, AUSTIN, TX, 78701, US

(21) Appl. No.: 12/355,400

(22) Filed: Jan. 16, 2009

Related U.S. Application Data
(60) Provisional application No. 61/021,498, filed on Jan. 16, 2008.

(51) Int. Cl. A61M 5/32

(52) U.S. Cl. 604/506; 604/264; 604/531

(57) ABSTRACT

A cannula device for insertion into a human or animal body, and method for using such cannula device, are disclosed, wherein all or part of the cannula is made of a shape memory alloy with an austenitic transformation temperature at or above the range at which medical instruments are sterilized. At working temperature, the shape memory alloy portion of the cannula is in a martensitic state and, therefore, pliable. The cannula is bent and shaped during use, either intentionally or unintentionally. After use, the shape memory alloy portion of the cannula may be placed in a conventional heat sterilization unit in which the temperature is equal to or above the austenitic transformation temperature causing the alloy to return to its memorized, substantially straight, austenitic configuration. After removal of the cannula from the heat sterilaization unit, the cannula is both sterilized and straightened to substantialy the original configuration and is thereby in condition for reuse.

### CROSS-REFERENCE TO RELATED APPLICATIONS

This non-provisional application claims priority based upon prior U.S. Provisional Patent Application Ser. No. 61/021498 filed Jan. 16, 2008 in the name of Kelly Tjelmeland, entitled “Reusable Cannula,” the disclosure of which is wholly incorporated herein by reference.

### FIELD OF THE INVENTION

This invention relates to a cannula and its method of use. More specifically, this invention relates to a cannula which is mechanically deformed from an initial configuration during use and, when sterilized through the application of heat, the cannula resumes its original configuration and is then in a condition for reuse.

### BACKGROUND OF THE INVENTION

Cannulas, including catheters, stents, and the like, are flexible or rigid tube-like devices generally made of metal or plastic. These devices are usually inserted into and passed through an incision, body orifice, peripheral artery, vein, or urogenital tract of a human or animal body until they reach the desired organ, structure, or cavity within the body. These devices are available in many forms and are used for a wide variety of purposes, including diagnostic and therapeutic purposes, and their particular construction tends to vary accordingly to the desired use.

Among the most common uses for cannula are: creating an opening for the viewing of tissue inside the body; injecting dyes and medicines into the body; removing fat through liposuction; sampling body fluids; monitoring the electrical properties of body organs such as the heart; creating a passageway for insertion of smaller diameter cannula; and others. However, many of these procedures require that the cannula be bent or shaped during use and the deformation causes them to be unsuitable for reuse.

For example, during liposuction, the removal of excess fat tissue is typically accomplished by inserting the distal end of a narrow metal cannula through a small incision in the skin and applying a vacuum suction, generally through a hose attached to the proximal end of the cannula. Liposuction cannulas generally consist of a hollow handle in which the shaft of the cannula is inserted. Various tip and hole configurations through which fat is suctioned are situated at the distal end of the cannula. After inserting the distal end of the cannula through the incision in the skin, the surgeon carefully moves the cannula forward and backward within the layer of fat. This movement shears off fat tissue particles which are drawn into the cannula and out of the body by the vacuum. During normal use, the cannula is bent to some degree intentionally or unintentionally. Over time the metal fatigues and breaks.

In another example, during the insertion of a cannula into a coronary artery, the cannula needs to be sufficiently stiff that it can be guided into place, yet at the same time sufficiently soft so as not to damage or penetrate the body tissue. The cannula often needs to navigate a substantially non-linear route in order to arrive at the desired location and, during the process, the cannula is bent and shaped causing it to lose its original form.

In yet another example, a cannula may be used during the infusion of intravenous fluids, in which case the distal end of a venous cannula is inserted into a vein and a fluid is then passed through the cannula. During major operations and in critical care areas, an arterial cannula may be inserted into an artery, commonly the radial artery, and is used to measure beat-to-beat blood pressure and to draw repeated blood samples.

In each of the aforementioned cases, it may be necessary to bend or shape the cannula, in which case the device will be deformed with shapes and curves that make it difficult or impossible to reuse for its intended use. In addition, even if a used cannula is sterilized, the bends and curves give the apearance that the devise is unsanitary and the deformaties make storage and identification difficult.

### BRIEF SUMMARY OF THE INVENTION

The present invention provides a cannula for insertion into the body of an individual or animal, comprising a shape memory alloy portion having an austenitic and a martensitic state, an austenitic transformation temperature which is at or above the range at which medical instruments are sterilized, and being transformable between the martensitic state to the austenitic state. In one embodiment, the cannula has an initial configuration in which it is substantially straight and the shape memory alloy portion of the cannula is in a martensitic state and, therefore, pliable. The cannula is then shaped or bent, either intentionally or unintentionally, during use. After use, the shape memory alloy portion of the cannula may be placed in a conventional heat sterilization unit in which the temperature is equal to or above the austenitic transformation temperature causing the alloy to return to its memorized, substantially straight, austenitic configuration. After removal of the cannula from the heat sterilaization unit, the cannula is both sterilized and straightened to substantialy the original configuration and is thereby in condition for reuse.

### BRIEF DESCRIPTION OF THE DRAWINGS

FIG. 1 shows the relationship between temperature and the percent austentite in a shape memory alloy;

FIG. 2 shows a plan view of a liposuction cannula prior to use;

FIG. 3 shows a plan view of a liposuction cannula after use;

FIG. 4 shows a plan view of a liposuction cannula after heat sterilization;

FIG. 5 shows a plan view of a curvilinear liposuction cannula prior to use;

FIG. 6 shows a plan view of a curvilinear liposuction cannula after use; and

FIG. 7 shows a plan view of a curvilinear liposuction cannula after heat sterilization.

### DETAILED DESCRIPTION

Alloys that display a shape memory effect, sometimes called “shape memory alloys,” are known in the art. In general, shaping and heat-treating a shape memory alloy causes the alloy to memorize its shape. Thereafter, when the alloy is shaped or bent at a temperature lower than its transformation point, the shape of the alloy is changed, but when the alloy is heated to its transformation point, it instantaneously restores its original shape. For example, if the alloy is shaped and heat-treated in a straight configuration and it is subsequently bent at a temperature lower than its transformation point, the original straight shape is instantaneously restored when the alloy is heated to its transformation point. An alloy's transformation point may be modified by adjusting the composition of the alloy or the heat treatment process.

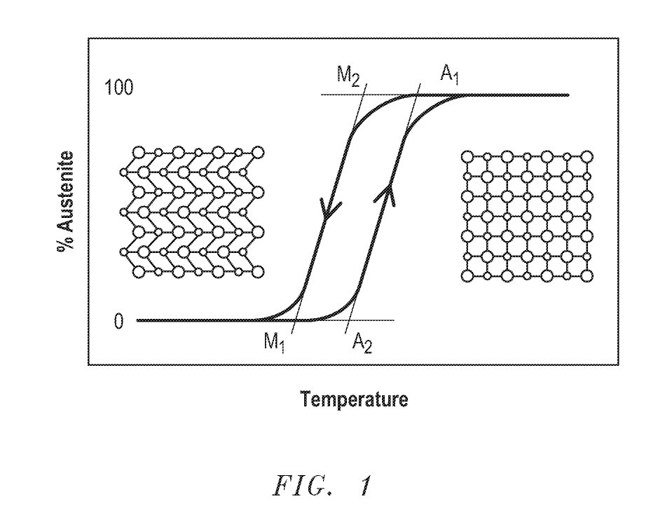

A shape memory alloy's ability to remember its original configuration is the result of a metallurgical phase transformation. Certain shape memory alloys are characterized by a transition temperature above which the predominant metallurgical phase is termed “austenite” and below which the predominant metallurgical phase is termed “martensite.” The transformation from the austenitic state to the martensitic state is termed the “martensitic transformation” and the transformation from martensitic state to the austenitic state is termed an “austenitic transformation.” This transformation is shown in FIG. 1, wherein, as the temperature increases, the percentage of austenite in the alloy increases from 0% at the initial temperature (As) to 100% at the final temperature (Af). Similarly, during cooling the percentage of martensite in the alloy decreases from 0% at the initial (higher) temperature (Ms) and decreases to 100% at the final (lower) temperature (Mf). These range where these transformations occur is called the transformation temperature range. The transformation between these two phases is reversible wherein the alloys may assume different properties in each of the two phases and can move back and forth between the phases such that each phase retains its own separate properties.

Shape memory materials are generally relatively pliable when the material is at a temperature below the transformation temperature range (i.e. the martensite range) and relatively strong with superelastic properties when the material is at a temperature above the transformation temperature range (i.e. austenite range). In other words, in the martensitic state, the alloy is malleable and bendable and, when bent, will stay bent and, in the austenitic state, the alloy is superelastic, stiff and spring-like and will return to its original shape. Within the transformation temperature range, the properties of a shape memory material typically vary but, in general, the strength and superelastic characteristics tend to increase toward the high temperature end of the transformation temperature range and decrease toward the low temperature end. Alloy compositions can be manipulated so that a normal body temperature is above, within, or below the transformation temperature range.

The alloy used in accordance with the invention may be any of a variety of shape memory alloys known in the art. Nitinol, an alloy comprising about 50 atomic percent of both nickel and titanium, is generally preferred. A suitable alloy composition includes about 30% to about 52% titanium, about 38% to about 52% nickel. If desired, up to about 20% copper or about 10% of other alloying elements may be added. The additional alloying elements may be selected from the group consisting of iron, cobalt, chromium, platinum, palladium, zirconium, hafnium, niobium and vanadium. All references to percent alloy compositions are atomic percent unless otherwise noted. The composition is selected within the ranges described above along with the thermomechanical processing variables for forming the cannula to provide a desired final austenite transformation temperature. Where all or part of the cannula is to be in the martensite phase at body temperature, the composition should be approximately equal atomic percentages of nickel and titanium.

Cannulas, including catheters, stents, and the like, are used in a wide variety of medical procedures. They are generally inserted into, and passed through, an incision, body orifice, peripheral artery, vein, or urogenital tract of a human or animal body until they reach the desired organ, structure, or cavity within the body. For example, a liposuction cannula is conventionally a thin tube with an aspirator tip at the distal end. The aspirator tip may include small openings at the sides or end of the tip and is designed to create passages between the tissue and the central lumen of the cannula. The central lumen is then in fluid communication with a suction source so that tissue and fluids can be aspirated or suctioned through the cannula and into the tissue canister. The suction causes the tissue to be pulled into the openings at the aspirator tip of the cannula. As such, the cannula servers two purposes; namely, the cannula is used to crush, tear, or avulse the fatty tissue and then suction the fatty tissue through the central lumen to aspirate the tissue fragments and fluids from the operative site. The crushing, tearing and avulsing of the fatty tissue can sometimes cause the cannula to mechanically deform.

Medical instruments that enter an already sterile part of the body, such as in the blood or beneath the skin, must have a high sterility assurance level. One method of sterilizing medical products is through heat sterilization. There are a variety of techniques which include or employ the use of heat to sterilize products, and the temperature varies with each method. For example, gas sterilization occurs at or around 40 to 55° C., boiling sterilization occurs at or around 100° C., high-pressure steam sterilization occurs at or around 120° C., and dry-heat sterilization occurs at or around 200-250° C.

The present invention provides a cannula for insertion into the body of an individual or animal, comprising a shape memory alloy portion having an austenitic and a martensitic state, an austenitic transformation temperature which is at or above the range at which medical instruments are sterilized, and being transformable between the martensitic state to the austenitic state. In one embodiment, the cannula as an initial configuration in which it is substantially unbent. The cannula of the present invention is used in the same manner as in the prior art. For example, excess fat tissue is removed by inserting the distal end of a liposuction cannula through a small incision in the skin and applying a vacuum suction through a hose attached to the proximal end of the cannula. During use, the shape memory alloy portion of the cannula is in a martensitic state and, therefore, pliable. After use, the shape memory alloy portion of the cannula may be placed in a conventional heat sterilization unit in which the temperature is equal to or above the austenitic transformation temperature causing the alloy to return to its memorized, substantially straight, austenitic configuration. After removal of the cannula from the heat sterilaization unit, the cannula is both sterilized and straightened to substantialy the original configuration and is thereby in condition for reuse.

Referring now to the drawings, and more particularly to FIG. 2, there is shown a plan view of a cannula generally depicted as reference numeral 2. The cannula 2 includes a tube 4 having a hollow lumen 6 along a length thereof. The hollow tube 4 includes an aspirator tip 8 at the distal end thereof having openings 10 for the aspiration of fat tissue and other debris and liquid from a surgical site. The tube 4 is made, at least in part, of a shape memory alloy having an austenitic transformation temperature which is at or above the range at which medical instruments are sterilized. At the proximal end of the tube 4 is a hub or connector assembly 12 which is mounted to a surgical tool and a suctioning device (not shown). The hub or connector assembly 12 may also represent a handle for use in manual systems. The suctioning device may be a vacuum or syringe used to provide a suctioning of the fat tissue, debris and the like from the surgical site via the openings 16 and bore of the hollow tube 12.

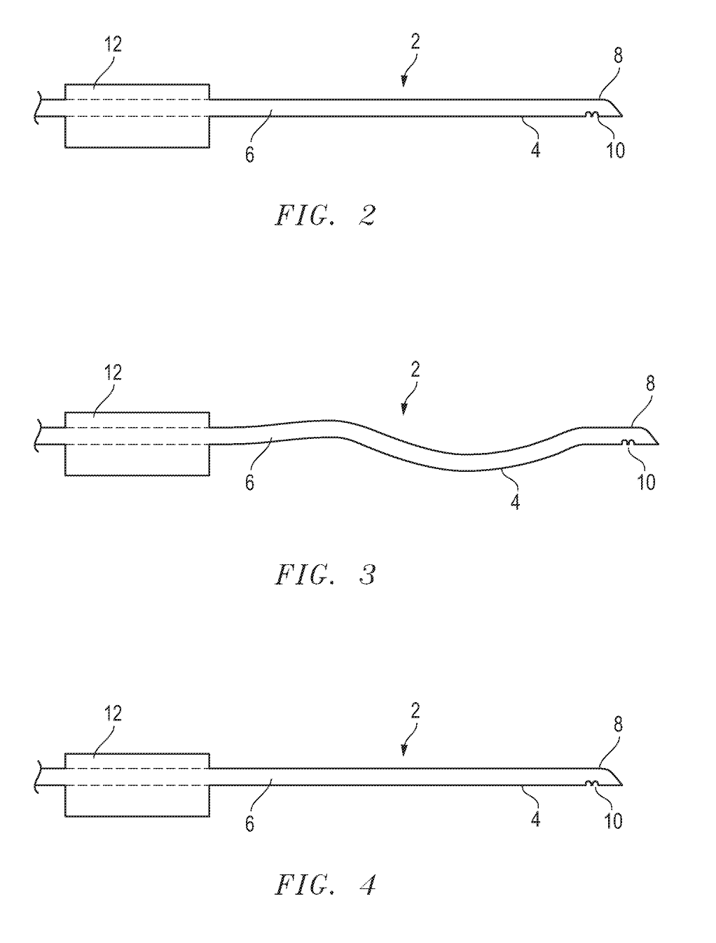

FIG. 3 shows a plan view of a liposuction cannula after use. Because of the manipulation required during suctioning, the tube 4 is mechanically deformed. In some circumstances, the mechanical deformations can be relatively small and under other circumstances they can be substantial. In either case, the used cannula 2 is not in a condition for reuse due to the deformation of the tube 4.

FIG. 4 shows a plan view of the liposuction cannula after it has been heat sterilized. Because the temperature required for heat sterilization is above the austenitic transformation temperature of the shape memory alloy used in the cannula, the cannula reverts to its original shape after heating.

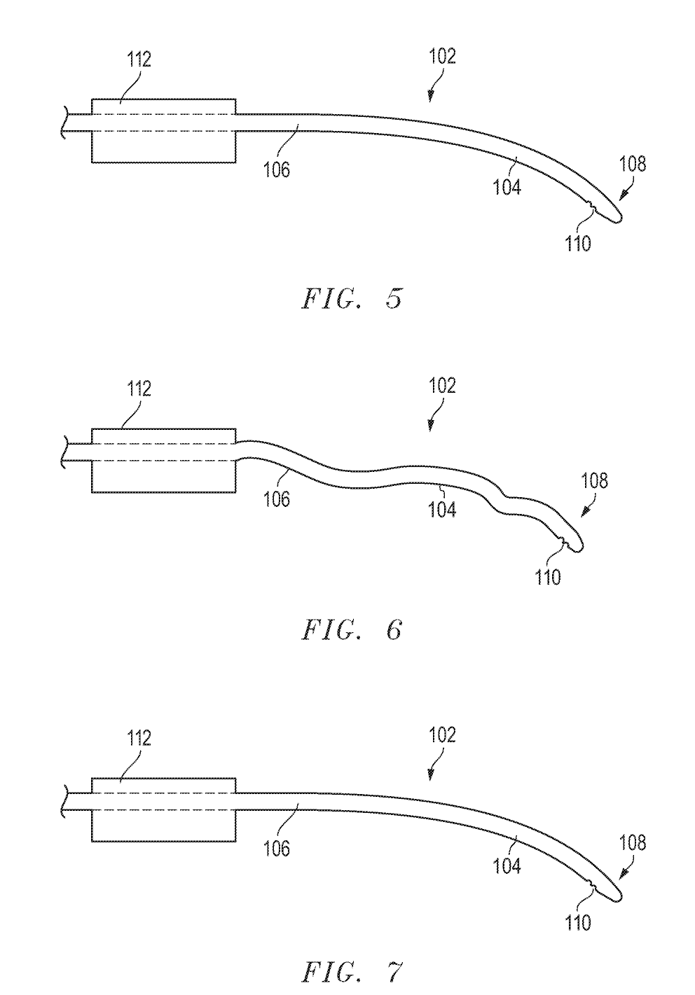

FIG. 5 shows a plan view of a cannula that is curvilinear in its original configuration. Once again, cannula 102 includes a tube 104 having a hollow lumen 106 along a length thereof and the tube 4 is made, at least in part, of a shape memory alloy having an austenitic transformation temperature which is at or above the range at which medical instruments are sterilized.

FIG. 6 shows a plan view of the same liposuction cannula after use with multiple deformities. FIG. 7 shows a plan view of the liposuction cannula after it has been heat sterilized. Once again, because the temperature required for heat sterilization is above the austenitic transformation temperature of the shape memory alloy used in the cannula, the cannula reverts to its original curvilinear shape after heating.

It will be apparent to those skilled in the art that some or all of the cannula may be made with the shape memory alloy. In one embodiment, only the functional portion of the cannula, that is, the portion of the cannula that performs the desired function within the body, is made from the shape memory alloy. In other embodiments, the entire cannula as well as the handle are made from the shape memory alloy. The tip of the cannula may be wholly integrated into the cannula or may be removably attached thereto.

In other embodiments, the portion of the cannula which inserts into the handle can be made of an alloy which is in the austenite state at working temperatures for a short distance up to an inch or two as it exits the handle to keep the cannula from bending constantly at the handle/cannula junction.

In yet another embodiment, the distal portion of the cannula near the tip can be made of an alloy that is in its austenite state at working temperatures. Thus the likely finished product will be malleable from a short distance from the handle to right before the suction holes.

While the present system and method has been disclosed according to the preferred embodiment of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise.

When a single embodiment is described herein, it will be readily apparent that more than one embodiment may be used in place of a single embodiment. Similarly, where more than one embodiment is described herein, it will be readily apparent that a single embodiment may be substituted for that one method or device.

In light of the wide variety of possible methods for making and using cannulas, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

What is claimed is:

1. A reusable medical device comprising
a cannula portion, wherein all or part of said cannula is made from a shape memory alloy having an austenitic transformation temperature which is at or above the range at which medical instruments are sterilized; said cannula being deformed during use while in its martensitic state and, thereafter, said cannula being heat sterilized resulting in said cannula returning substantially to its original configuration.

2. The cannula of claim 1, wherein said cannula is a catheter or a stint.

3. The cannula of claim 1, wherein said shape memory alloy is about 50% nickel and about 50% titanium.

4. The cannula of claim 1, wherein said cannula is sterilized using gas sterilization, boiling sterilization, high pressure steam sterilization or dry heat sterilization.

5. The cannula of claim 1, wherein a handle is affixed to said cannula and the portion of said cannula proximate to said handle is made of an alloy which is in its austenitic state at working temperature and another portion of said cannula is made from an alloy that is in its martensitic state at working temperature.

6. A method of using a cannula comprising:
inserting a cannula into a body, all or part of said cannula being made from a shape memory alloy having an austenitic transformation temperature which is at or above the range at which medical instruments are sterilized;
manipulating said cannula during use resulting in deformation thereof;
sterilizing said cannula, wherein said sterilization results in said cannula returning substantially to its original configuration.

7. The method of claim 6 wherein said cannula is used during a liposuction procedure.

8. The method of claim 6, wherein said cannula is a catheter or a stint.

9. The method of claim 6, wherein said shape memory alloy is about 50% nickel and about 50% titanium.

10. The method of claim 6, wherein said cannula is sterilized using gas sterilization, boiling sterilization, high pressure steam sterilization or dry heat sterilization.

11. The method of claim 6, wherein a handle is affixed to said cannula and the portion of said cannula proximate to said handle is made of an alloy which is in its austenitic state at working temperature and another portion of the cannula is made from an alloy that is in its martensitic state at working temperature.
